(19) United States
(12) Patent Application Publication
(10) Pub. No.: US 2025/0160683 A1
(43) Pub. Date: May 22, 2025

(54) PATIENT SUPPORT APPARATUS WITH DYNAMIC WEIGHT EXIT DETECTION SYSTEM

(71) Applicant: Stryker Corporation, Kalamazoo, MI (US)

(72) Inventors: Sujay Sukumaran, Portage, MI (US); Madhu Thomas, London, Ontario (CA); Berkay Güncan, Talas, Kayseri (TR)

(21) Appl. No.: 18/724,691

(22) PCT Filed: May 17, 2023
(86) PCT No.: PCT/US2023/022545

§ 371 Date: Jun. 27, 2024

Related U.S. Application Data
(60) Provisional application No. 63/343,821, filed on May 19, 2022.

(51) Int. Cl. A61B 5/11; A61B 5/00; A61G 7/05; G01G 19/44; G01G 19/52; G01G 23/18

(52) U.S. Cl. CPC A61B 5/1115; A61B 5/6892; A61B 5/746; A61G 7/0527; G01G 19/445; G01G 19/52; G01G 23/18; A61G 2203/20; A61G 2203/32

(57) ABSTRACT

A patient support apparatus, such as a bed, cot, stretcher, etc., for a supporting a patient includes an exit detection system adapted to issue an alert when the patient exits from the patient support apparatus. When the exit detection system is armed, a controller onboard the patient support apparatus repetitively determines a current weight supported thereon, selects a variable threshold, compares the current weight to the variable threshold, and issues an exit alert if the current weight is less than the variable threshold. The variable threshold may be selected based on the output of a sensor, which in turn may measure a height of a litter frame of the patient support apparatus and/or the activation of an equipment log control on the patient support apparatus. The controller may be configured to determine a baseline weight reading and to select the variable threshold as a percentage of the baseline weight reading.

### BACKGROUND

The present disclosure relates to patient support apparatuses, such as beds, cots, stretchers, operating tables, recliners, or the like. More specifically, the present disclosure relates to patient support apparatuses that include an exit detection system that issues an alert when the patient exits the patient support apparatus.

Existing hospital beds and/or stretchers often include an exit detection system that is adapted to detect when a patient has exited the bed, or when a patient may be about to exit the bed. Typically, such beds include circuitry for providing an audio or visual alert when such an exit or pre-exit situation is detected. In many cases, the bed or stretchers include circuitry for transmitting a signal to a remote location, such as a nurses' station, so that the appropriate caregivers are notified of the exit, or pre-exit condition, and can respond appropriately. The exit detection system itself may be implemented in a variety of manners, including using a plurality of force sensors. In general, it is desirable to implement the exit detection system so as to minimize false alerts, but also prevent the patient from being able to exit the bed without an alert being issued while the exit detection system is armed.

### SUMMARY

According to various embodiments, an improved patient support apparatus is provided that helps reduce false alerts and/or helps reduce the ability of a patient to exit without detection by the exit detection system when the exit detection system is armed. In some embodiments, the exit detection system is adapted to dynamically change a weight threshold that is used as a trigger for issuing the exit alert. The dynamic changing of the weight threshold may be based upon the state of one or more components of the patient support apparatus that are capable of negatively affecting the exit detection system. The changes to the weight threshold may be implemented so as to counteract those potential negative effects. In some aspects, the dynamic weight threshold refers to the drop in weight that must be detected by the exit detection system in order to trigger an exit alert. Still other features will be apparent to those skilled in the art in light of the following description and accompanying claims.

According to one aspect of the present disclosure, a patient support apparatus is provided that includes a litter frame, a support surface, a plurality of force sensors, a control panel, a sensor, and a controller. The support surface is supported by the litter frame and is adapted to support a patient thereon. The plurality of force sensors are adapted to detect downward forces exerted on the support surface. The control panel is adapted to allow a user to arm and disarm an exit detection function. The sensor is adapted to detect a state of component of the patient support apparatus. The controller is adapted, when the exit detection function is armed, to repetitively determine a current weight supported on the support surface using outputs from the plurality of force sensors, to select a variable threshold based on an output from the sensor, to compare the current weight to the variable threshold, and to issue an exit alert if the current weight is less than the variable threshold.

According to other aspects of the present disclosure, the controller may be further adapted to determine a baseline weight reading when the patient is supported on the patient support apparatus using outputs from the plurality of force sensors, and to select the variable threshold by selecting a percentage of the baseline weight reading.

In some aspects, the patient support apparatus further includes a lift subsystem adapted to raise and lower the litter frame, and the output of the sensor is indicative of a height of the litter frame.

The controller, in some aspects, is adapted to select a first value for the variable threshold when the height of the litter frame is at a low height and to select a second value for the variable threshold when the height of the litter frame is at a high height. The first value is higher than the second value.

The patient support apparatus, in some aspects, also includes an equipment change control adapted to be activated by a user in order to update an onboard log of equipment weight. The controller may be adapted to use the onboard log of equipment weight to keep track of which component of the current weight belongs to the patient and which component belongs to equipment.

The output of the sensor, in some aspects, is indicative of the user activating the equipment change control.

The controller, in some aspects, is adapted to select a first value for the variable threshold when the equipment change control have been activated and to select a second value for the variable threshold when the equipment change control has not been activated. The first value is less than the second value.

The equipment change control, in some aspects, includes an icon on a touchscreen display and the sensor is a touch sensor.

The patient support apparatus, in some aspects, further includes a sensitivity control adapted to allow a user to select a first sensitivity level or a second sensitivity level for issuing the exit alert. The controller is adapted to compare the current weight to the variable threshold when the user has selected the first sensitivity level, but not when the user has selected the second sensitivity level.

In some aspects, the controller is adapted to compare the current weight to a fixed threshold when the user has selected the second sensitivity level, and to issue the exit alert if the current weight is less than the fixed threshold.

The controller, in some aspects, is further adapted to repetitively calculate a center of gravity of the patient using outputs from the plurality of force sensors, to repetitively compare the calculated center of gravity to a zone, and to issue the exit alert if the calculated center of gravity moves outsides of a boundary of the zone.

The boundary of the zone, in some aspects, is a rectilinear boundary having at least seven sides.

In some aspects, the boundary of the zone defines a shape having a first width defined toward a head end of the patient support apparatus and a second width defined toward a foot end of the patient support apparatus, and the first width is greater than the second width.

According to another aspect of the present disclosure, a patient support apparatus is provided that includes a litter frame, a support surface, a plurality of force sensors, a control panel, a sensitivity control, and a controller. The support surface is supported by the litter frame and adapted to support a patient thereon. The force sensors are adapted to detect downward forces exerted on the support surface. The control panel is adapted to allow a user to arm an exit detection function. The sensitivity control is adapted to allow a user to select a first sensitivity level or a second sensitivity level for issuing an exit alert when the exit detection function is armed. The controller is adapted to—when the exit detection function is armed and the first sensitivity level is selected—perform the following: (a) repetitively determine a current weight supported on the support surface using outputs from the plurality of force sensors; (b) compare the current weight to a first threshold; and (c) issue the exit alert if the current weight is less than the first threshold. The controller is also adapted to—when the exit detection function is armed and the second sensitivity level is selected-perform the following: (i) repetitively determine the current weight supported on the support surface using outputs from the plurality of force sensors; (ii) compare the current weight to a second threshold; and (iii) issue the exit alert if the current weight is less than the second threshold. The first threshold is different from the second threshold.

According to other aspects of the present disclosure, the patient support apparatus may further comprise a sensor adapted to detect a state of a component of the patient support apparatus, and wherein the controller is further adapted to vary the first threshold based on an output of from the sensor.

In some aspects, the patient support apparatus further includes a lift subsystem adapted to raise and lower the litter frame, and the output of the sensor is indicative of a height of the litter frame.

The controller, in some aspects, is adapted to select a first value for the first threshold when the height of the litter frame is at a low height and to select a second value for the first threshold when the height of the litter frame is at a high height, wherein the first value is higher than the second value.

The patient support apparatus, in some aspects, includes an equipment change control adapted to be activated by a user in order to update an onboard log of equipment weight, wherein the controller is adapted to use the onboard log of equipment weight to keep track of which component of the current weight belongs to the patient and which component of the current weight belongs to equipment.

The sensor, in some aspects, produces an output that is indicative of the user activating the equipment change control.

In some aspects, the controller is adapted to select a first value for the first threshold when the equipment change control have been activated and to select a second value for the first threshold when the equipment change control has not been activated, wherein the first value is less than the second value.

The equipment change control, in some aspects, includes an icon on a touchscreen display and the sensor is a touch sensor.

The controller, in some aspects, is further adapted to determine a baseline weight reading when the patient is supported on the patient support apparatus using outputs from the plurality of force sensors, and to select the first threshold by selecting a percentage of the baseline weight reading.

The controller, in some aspects, is further adapted to repetitively calculate a center of gravity of the patient using outputs from the plurality of force sensors and, when the exit detection function is armed and the first sensitivity level is selected, perform the following: (d) repetitively compare the calculated center of gravity to a first zone, and (e) issue the exit alert if the calculated center of gravity moves outsides of a boundary of the first zone. When the exit detection function is armed and the second sensitivity level is selected, the controller is further adapted to perform the following: (iv) repetitively compare the calculated center of gravity to a second zone, and (v) issue the exit alert if the calculated center of gravity moves outsides of a boundary of the second zone.

In some aspects, the boundary of the first zone is a rectilinear boundary having at least seven sides, and the boundary of the second zone is a rectangle.

The boundary of the first zone, in some aspects, defines a shape having a first width defined toward a head end of the patient support apparatus and a second width defined toward a foot end of the patient support apparatus, and the first width is greater than the second width.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to one aspect of the disclosure;

FIG. 2 is a diagram of a control system of the patient support apparatus of FIG. 1, as well as several external devices with which the patient support apparatus may be configured to communicate;

FIG. 3 is a plan view of a control panel of the patient support apparatus of FIG. 1;

FIG. 4 is an arming screen displayable on the control panel of the FIG. 3 that is adapted to allow a user to arm and disarm an exit detection system of the patient support apparatus;

FIG. 5 is a diagram illustrating a reference frame for the patient support apparatus and the manner in which multiple user-selectable modes of the exit detection system map to the reference frame;

FIG. 6 is a diagram of a plurality of exit detection zones that may be used by the exit detection system when implementing the multiple user-selectable mode;

FIG. 7 is a flow chart illustrating an exit detection operation algorithm of the patient support apparatus; and

FIG. 8 is scale control screen displayable on the control panel that includes an equipment weight log control.

### DETAILED DESCRIPTION OF THE EMBODIMENTS

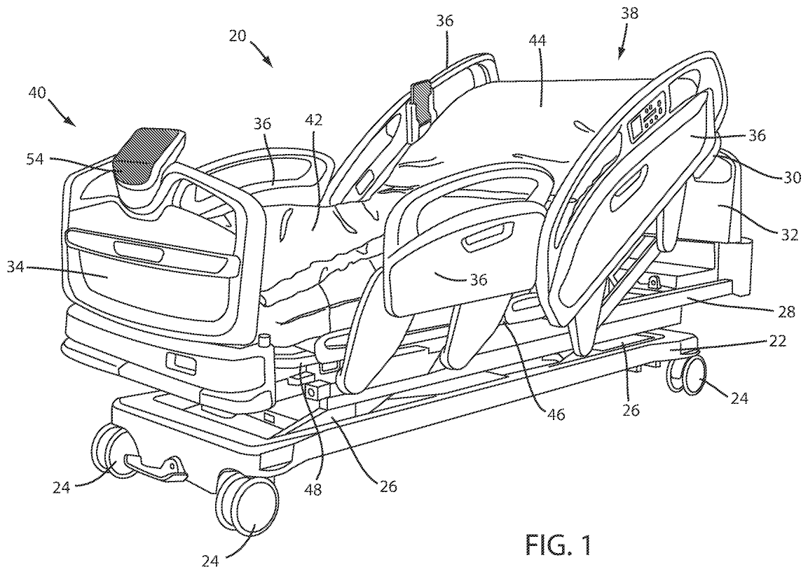

An illustrative patient support apparatus 20 that may incorporate one or more aspects of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a gurney, a recliner, a residential bed, or any other structure capable of supporting a patient, whether stationary or mobile and/or whether medical or residential.

In general, patient support apparatus 20 includes a base 22 having a plurality of wheels 24, a pair of lifts 26 supported on the base, a litter frame 28 supported on the lifts 26, and a support deck 30 supported on the litter frame 28. Patient support apparatus 20 further includes a headboard 32, a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. The top surface of the mattress 42 or other cushion forms a support surface for the occupant.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width. Notionally, the first standard width may be considered a 36 inch width, the second intermediate width may be considered a 42 inch width and the third more expanded width may be considered a 48 inch width, although these numerical widths may be varied to comprise different width values.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may be the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

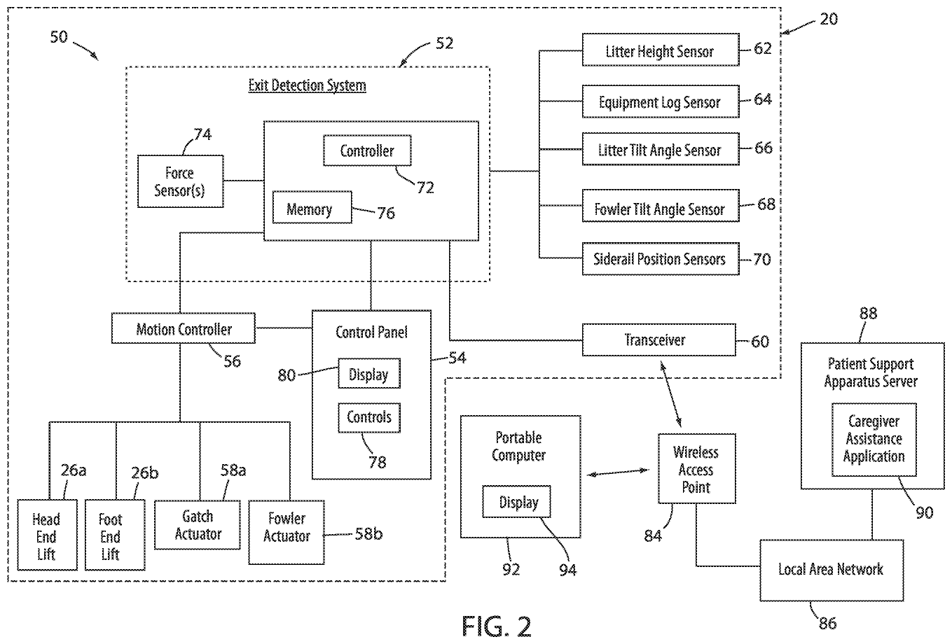

As shown in FIG. 2, patient support apparatus 20 includes a control system 50 that controls various aspects of patient support apparatus 20. Control system 50 includes an exit detection system 52, at least one control panel 54, a motion controller 56, lifts 26a and 26b, a plurality of motorized actuators 58a and 58b, an off-board communications transceiver 60, a litter frame height sensor 62, an equipment log sensor 64, a litter tilt angle sensor 66, a Fowler tilt angle sensor 68, and a plurality of siderail position sensors 70.

Exit detection system 52 includes a controller 72, a plurality of force sensors 74, and a memory 76. Exit detection system 52 is adapted to determine when an occupant, such as, but not limited to, a patient, of patient support apparatus 20 is likely to exit patient support apparatus 20. More specifically, exit detection system 52 is adapted to determine when an occupant is likely to leave prior to the occupant actually leaving, and to issue an exit alert and/or notification to appropriate personnel so that proper steps can be taken in response to the occupant's imminent departure in a timely fashion. The particular structural details of exit detection system 52 can vary widely from what is shown in FIG. 2.

Force sensors 74 are adapted to detect downward forces exerted by an occupant of support deck 30. Thus, when an occupant is positioned on support deck 30 and remains substantially still (i.e. not moving in a manner involving accelerations that cause forces to be exerted against support deck 30), force sensors 74 will detect the weight of the occupant (as well as the weight of any components of patient support apparatus 20 that are supported—directly or indirectly—by force sensors 74). In at least one embodiment, force sensors 74 are four load cells that are positioned in corners of a rectangular shape lying in a generally horizontal plane beneath the upper surface of support deck 30. However, it will be understood by those skilled in the art, that force sensors 74 may be implemented as other types of sensors, such as, but not limited to, linear variable displacement transducers and/or any one or more capacitive, inductive, and/or resistive transducers that are configured to produce a changing output in response to changes in the force exerted against them. Other locations of the force sensors 74, and/or other numbers of force sensors 74, may also be used.

Exit detection controller 72 is constructed of any electrical component, or group of electrical components, that are capable of carrying out the functions described herein. In many embodiments, controller 72 is a conventional microcontroller, although not all such embodiments need include a microcontroller. In general, controller 72 includes any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. Indeed, in some embodiments, exit detection controller 72 is combined with motion controller 56 and/or with one or more other controllers present on patient support apparatus 20. The instructions followed by controller 72 in carrying out the functions described herein, as well as the data necessary for carrying out these functions, are stored in memory 76, which is accessible to controller 72.

Although patient support apparatus 20 includes a total of four force sensors 74, it will be understood by those skilled in the art that different numbers of force sensors 74 may be used in accordance with the principles of the present disclosure. Force sensors 74, in at least one embodiment, are configured to support litter frame 28. When so configured, force sensors 74 are constructed to provide complete and exclusive mechanical support for litter frame 28 and all of the components that are supported on litter frame 28 (e.g. deck 30, headboard 32, footboard 34, and, in some embodiments, siderails 36). Because of this construction, force sensors 74 are adapted to detect the weight of not only those components of patient support apparatus 20 that are supported by the litter frame 28 (including litter frame 28 itself), but also any objects or persons who are positioned either wholly or partially on support deck 30. By knowing the weight of the components of the patient support apparatus 20 that are supported on litter frame 28, controller 72 is able to determine a tare weight that, when subtracted from a total weight sensed after a patient is supported on support deck 30, yields a patient weight.

In some embodiments, the physical location of the force sensors 74 on patient support apparatus 20 may be modified to be located on the base frame, such as shown in commonly assigned U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. In other embodiments, the physical location of the force sensors 74 on patient support apparatus 20 may be the same as the position of the load cells disclosed in commonly assigned U.S. patent application Ser. No. 15/266,575 filed Sep. 15, 2016, by inventors Anuj Sidhu et al. and entitled PERSON SUPPORT APPARATUSES WITH EXIT DETECTION SYSTEMS, the complete disclosure of which is also incorporated herein by reference. In still other embodiments, the physical location of the force sensors 74 may be the same as the position of the load cells disclosed in U.S. Pat. No. 7,962,981 issued to Lemire et al. and entitled HOSPITAL BED, the complete disclosure of which is also incorporated herein by reference. In still other embodiments, force sensors 74 may be positioned on patient support apparatus 20 at still other locations.

Motion controller 56 (FIG. 2) is adapted to control the movement of a plurality of components of patient support apparatus 20. These components includes, but are not limited to, head end lift 26a, foot end lift 26b, a gatch actuator 58a, and a Fowler actuator 58b. Each of these actuators 26a-b, 58a-b may comprise a linear actuator with a motor built therein. In some embodiments, the linear actuator may be of the type disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In other embodiments, other types of powered actuators may be used, such as, but not limited to, hydraulic and/or pneumatic actuators.

In some embodiments, motion controller 56 is a conventional microcontroller that controls the operation of the motors contained with each of actuators 26a-b, 58a-b. It will be understood that motion controller 56 may be constructed of any electrical component, or group of electrical components, that are capable of carrying out the functions described herein. In general, controller 56 includes any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. Indeed, in some embodiments, as noted above, motion controller 56 may be combined together with exit detection controller 72 and/or with one or more other controllers present on patient support apparatus 20. The instructions followed by controller 56 in carrying out the functions described herein, as well as the data necessary for carrying out these functions, are stored in a memory (not shown) that is accessible to controller 56.

In some embodiments, motion controller 56 operates in the same or similar manners to the main microcontroller 58 and its associated circuitry disclosed in commonly assigned U.S. Pat. No. 10,420,687 issued Sep. 24, 2019, to inventors Aaron Furman et al. and entitled BATTERY MANAGEMENT FOR PATIENT SUPPORT APPARATUSES, the complete disclosure of which is incorporated herein by reference. In such embodiments, motion controller 56 controls the sending of pulse width modulated (PWM) signals to the motors contained within actuators 26a-b, 58a-b, thereby controlling both the speed and the direction of movement of these actuators. Motion controller 56 may take on other forms as well.

Motion controller 56 is in communication with control panel 54 and receives signals from control panel 54 indicating when a user wishes to move one or more components of patient support apparatus 20. That is, control panel 54 includes one or more controls 78 that are adapted, when activated, to instruct motion controller 56 to carry out the desired movement of the various movable components of patient support apparatus 20, as well as one or more controls for stopping such motion. Such movement includes, but is not limited to, raising and lowering the height of litter frame 28, pivoting the Fowler section 44 up and down about a generally horizontal axis (extending laterally from one side of the patient support apparatus 20 to the other), and/or lifting and lowering a knee gatch on patient support apparatus 20.

Head end lift actuator 26a is configured to change the height of the head end 38 of litter frame 28. Foot end lift actuator 26b is configured to change the height of the foot end 40 of litter frame 28. When both of these actuators 26a and 26b are operated simultaneously and at the same speed, the height of litter frame 28 is raised or lowered without changing the general orientation of litter frame 28 with respect horizontal. When one or more of these actuators 26a and/or 26b are operated at different times and/or at different speeds, the orientation of litter frame 28 is changed with respect to horizontal. Lift actuators 26a and 26b are therefore able to tilt litter frame 28 to a variety of different orientations, including, but not limited to, a Trendelenburg orientation and a reverse-Trendelenburg orientation.

Gatch actuator 58a is adapted to raise and lower the joint that couples together the thigh section 46 and the foot section 48 of support deck 30, thereby raising and lowering the portion of the support deck 30 that is positioned close to the patient's knees. Fowler actuator 58b is adapted to raise and lower the head section (or Fowler section) 44 of the support deck 30.

Control panel 54 (FIG. 2) may be one of several control panels positioned on patient support apparatus 20. Although control panel 54 is show in FIG. 1 to be positioned on footboard 34, it will be understood that control panel 54 may be positioned at other locations, such as, but not limited to, on one or more of the siderails 36. Control panel 54 communicates with exit detection system 52 and motion controller 56 and includes a plurality of controls 78 that are adapted to control various aspects of patient support apparatus 20, including, but not limited to, exit detection system 52 and motion controller 56. The plurality of controls 78 may be implemented as buttons, dials, switches, icons on a touchscreen, or other devices. Control panel 54 also includes, in some embodiments, a display 80 for displaying information regarding exit detection system 52. Display 80 may be a touchscreen that displays one or more controls and/or one or more control screens, some of which are discussed in greater detail below. Display 80 may comprise an LED display, OLED display, or another type of display.

Control system 50 (FIG. 2) of patient support apparatus 20 also includes sensors 62-70, each of which is adapted to communicate information to exit detection system 52. Litter height sensor 62 is adapted to detect how high litter frame 28 has been lifted via lifts 26a-b. Alternatively, litter height sensor 62 may be a sensor that indicates whether or not litter frame 28 is currently positioned in its lowest height or not, and/or whether it is currently positioned in its highest height or not. Litter height sensor 62 reports the height of litter frame 28 to controller 72 of exit detection system 52.

Equipment log sensor 64 detects whether an equipment log maintained onboard patient support apparatus 20 is being modified by a user or not. In some embodiments, patient support apparatus 20 is configured to maintain an equipment log in memory 76 that maintains a running log of the weight of objects that have been added to patient support apparatus 20 after the scale system—which is integrated into the exit detection system 52—has been tared. Controller 72 uses this equipment log weight to separate the component of the weight sensed by force sensors 74 that is due to equipment (any non-patient objects) from the component of the weight sensed by force sensors 74 that is due to the patient. In this manner, controller 72 is able to determine an accurate patient weight, even after objects are added or removed, without requiring the patient to exit patient support apparatus 20 in order to re-tare the scale system. In some embodiments, the equipment log maintained by controller 72 may be the same as, or include any of the functions of, the equipment log disclosed in commonly assigned U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG, the complete disclosure of which is incorporated herein by reference. Equipment log sensor 64 reports to controller 72 whenever the user engages in the act of adding or removing equipment from the onboard equipment log. In some aspects, as will be discussed in greater detail below, equipment log sensor 64 is a touchscreen sensor that detects when the user has touched an icon on the touchscreen display that controls the equipment weight log.

Litter tilt angle sensor 66 detects the current angular orientation of litter frame 28 with respect to horizontal. Litter tilt angle sensor 66 detects this angle in the longitudinal direction along patient support apparatus 20 (e.g. in the direction from head end 38 to foot end 40, not in the side-to-side direction). Thus, litter tilt angle sensor 66 can be used to indicate whether litter frame 28 is in the Trendelenburg orientation, the reverse Trendelenburg orientation, or another orientation. In some embodiments, litter tilt angle sensor 66 detects the current angular orientation of litter frame 28 with respect to the base 22, rather than horizontal, in which case the output of sensor 66 will not vary when patient support apparatus 20 is positioned on an inclined or declined surface. In still other embodiments, litter tilt angle sensor 66 may comprise multiple sensors, such as, but not limited to, a first sensor that detects the angular orientation of litter frame 28 with respect to base 22 and a second sensor that detects the angular orientation of base frame 22 with respect to horizontal. Still other variations may be implemented, and regardless of the specific implementation, the output of the tilt sensor(s) are forwarded to exit detection system 52.

Fowler tilt angle sensor 68 detects the current angular orientation of Fowler section 44 with respect to horizontal (or, in some embodiments, with respect to the plane generally defined by litter frame 28 and/or the plane generally defined by support deck 30 when all of its sections are in a flat orientation). Fowler tilt angle sensor 68 reports its readings to exit detection system 52.

In some embodiments, one or both of litter tilt angle sensor 66 and/or Fowler tilt angle sensor 68 comprise position feedback sensors that are built into one or more of the actuators 26a-b, and/or 58a. In such embodiments, the sensors 66 and/or 68 may comprise Hall Effect sensors, encoders, and/or switches built into the actuators 26a-b and/or 58b that indicate the position of the extendable arm of the linear actuators. From this information, the angle of litter frame 28 and/or Fowler section 44 can be determined (such as by motion controller 56 and/or exit detection controller 72). Examples of such switches, sensors, and/or encoders are disclosed in the aforementioned commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, which is incorporated herein in its entirety.

Siderail position sensors 70 detect the position of each of the siderails 36 (up, down, or some other intermediate position) and report the positions of each of the siderails 36 to exit detection system 52.

All of the sensors 62-70 are adapted to communicate their information to exit detection system 52 which, as will be discussed in more detail below, may be configured to use some or all of this information when carrying out the exit detection function of exit detection system 52.

Transceiver 60 (FIG. 2) is adapted to communicate with one or more devices positioned off-board patient support apparatus 20, such as, but not limited to, a hospital's local area network. In some embodiments, transceiver 60 is a conventional WiFi transceiver (i.e. IEEE 802.11 . . . ) adapted to wirelessly communicate with one or more wireless access points 84 of a hospital's local area network 86. In other embodiments, transceiver 60 may be a conventional Ethernet transceiver electrically coupled to a conventional Ethernet port (i.e. RJ-45 jack, or the like) built into patient support apparatus 20 that allows a conventional Ethernet cable to be coupled to the patient support apparatus 20. In these embodiments, patient support apparatuses 20 may be coupled to the hospital's local area network 86 by a wired connection. In still other embodiments, patient support apparatus 20 may have both wired and wireless transceivers 60. Still further, in some embodiments, transceiver 60 may take on a different form (e.g. a wireless ZigBee transceiver, a Bluetooth transceiver, etc.).

Patient support apparatus 20 uses transceiver 60, in some embodiments, to communicate with a patient support apparatus server 88. Patient support apparatus server 88 may be adapted to receive status information from patient support apparatuses 20 and distribute that information to one or more other servers and/or other devices coupled to local area network 86. In at least one embodiment, patient support apparatus server 88 includes a caregiver assistance application 90 that is adapted to communicate information between both patient support apparatuses 20 and one or more portable electronic devices 92. The portable electronic devices 92 includes, but are not limited to, smart phones, tablets, laptops, Computers on Wheels (COWs), and the like. Each portable electronic device 92 includes a display 94 on which one or more of the screens discussed in more detail below may be displayed. In some embodiments, caregiver assistance application 90 allows authorized users to remotely configure and remotely control various aspects of the exit detection system 52 of patient support apparatuses 20 using their portable computing device 92. In some of such embodiments, caregiver assistance application 90 may be configured to operate in the same manner as, and/or may be configured to include any of the same functionality as, the caregiver assistance application disclosed in commonly assigned U.S. patent application Ser. No. 62/868,947 filed Jun. 30, 2019, by inventors Thomas Durlach et al. and entitled CAREGIVER ASSISTANCE SYSTEM, the complete disclosure of which is incorporated herein by reference. In such embodiments, patient support apparatuses 20 may include any of the components and/or functionality of the patient support apparatuses disclosed in the aforementioned '947 application; portable computing devices 92 may include any of the components and/or functionality of the electronic devices 104a and/or 104b disclosed in the aforementioned '947 application; and caregiver assistance application 90 may communicate with any one or more of the servers disclosed in the aforementioned '947 application.

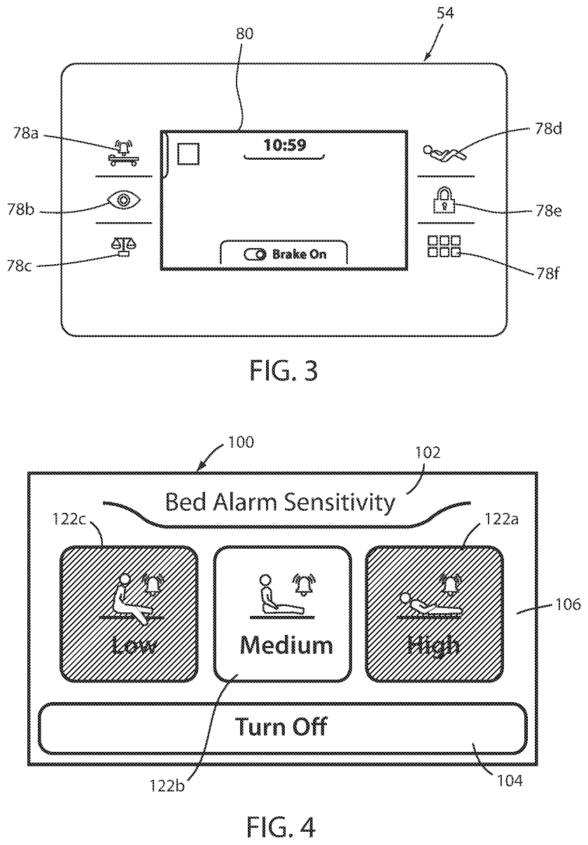

One example of control panel 54 is shown in FIG. 3. In this example, control panel 54 includes six dedicated controls 78a-f, three of which are positioned to the left of display 80 and three of which are positioned to the right of display 80. When a user presses navigation control 78a, control panel 54 displays an exit detection control screen on display 80 that includes one or more icons that, when touched, control an onboard exit detection system. The exit detection system is adapted to issue an alert when a patient exits from patient support apparatus 20. Such an exit detection system may include any of the same features and/or functions as, and/or may be constructed in any of the same manners as, the exit detection systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and/or the exit detection system disclosed in commonly assigned U.S. Pat. No. 5,276,432 issued to Travis and entitled PATIENT EXIT DETECTION MECHANISM FOR HOSPITAL BED, the complete disclosures of both of which are incorporated herein by reference. In some embodiments, controller 72 is configured to display an exit detection system arming screen 100 of the type shown in FIG. 4, as will be discussed in greater detail below.

When a user presses navigation control 78b (FIG. 3), control panel 54 displays a patient support apparatus monitoring control screen that includes a plurality of control icons that, when touched, control an onboard monitoring system that monitors one or more components, features, and/or other aspects of patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20 for monitoring parameters of the patient support apparatus 20.

When a user presses navigation control 78c, control panel 54 displays a scale control screen that includes a plurality of control icons that, when touched, control the scale system of patient support apparatus 20. Such a scale system may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors 74 that are utilized by the exit detection system, in some embodiments, or it may utilize one or more different sensors. Further details regarding the scale system are described in greater detail below.

When a user presses navigation control 78d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 80 in response to pressing control 78d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 78e (FIG. 3), control panel 54 displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 78f, control panel 54 displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, location information, medical device association information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20.

For all of the navigation controls 78a-f (FIG. 3), screens other than the ones specifically mentioned above may be displayed on display 80 in other embodiments of patient support apparatus 20 in response to a user pressing these controls. Thus, it will be understood that the specific screens mentioned above are merely representative of the types of screens that are displayable on display 80 in response to a user pressing on one or more of navigation controls 78a-f. It will also be understood that, although navigation controls 78a-f have all been illustrated in the accompanying drawings as dedicated controls that are positioned adjacent display 80, any one or more of these controls 78a-f could alternatively be touchscreen controls that are displayed at one or more locations on display 80. Still further, although controls 78a-f have been shown herein as buttons, it will be understood that any of controls 78a-f could also, or alternatively, be switches, dials, or other types of non-button controls.

FIG. 4 illustrates one example of an exit detection system arming screen 100 that is displayable on display 80 of control panel 54. As was noted, in some embodiments, arming screen 100 is displayed automatically in response to a user pressing exit detection control 78a. In other embodiments, arming screen 100 may be displayed in response to other triggers and/or be accessed by a user in other manners. Regardless of how accessed, exit detection system arming screen 100 includes an upper status bar 102 indicating (in this example) that exit detection system 52 has a plurality of sensitivity levels that the user can select from. Arming screen 100 also includes a lower control bar 104 that, when touched, allows a user to disarm exit detection system 52; and a sensitivity indicator region 106 that indicates what user-selectable sensitivity level the exit detection system 52 is capable of operating in (as well what level, if any, it is currently operating in).

In the embodiment discussed herein, exit detection system 52 is configured to operate with any one of three user-selectable sensitivity levels. It will be understood, however, that exit detection system 52 may be configured to operate in fewer user-selectable sensitivity levels or a greater number of user-selectable sensitivity levels, and that the following description of three user-selectable sensitivity levels is merely for purposes of illustrating the principles of the present disclosure.

The different user-selectable sensitivity levels refer to the relative freedom of movement of the patient when positioned on support deck 30. That is, a higher sensitivity level means that exit detection system 52 will issue an exit alert when the patient moves by a relatively small amount toward either side of the patient support apparatus 20 (and/or, in some embodiments, toward the head end 38 and/or the foot end 40 of patient support apparatus 20). When exit detection system 52 operates with a medium sensitivity level, the patent is free to move closer to the sides and/or ends of the patient support apparatus 20 than he or she is able to do so with the high sensitivity level without exit detection system 52 issuing an exit alert. When exit detection system 52 operates with a low sensitivity level, the patient is the most free to move toward the sides and/or ends of the patient support apparatus without triggering an exit detection alert.

In some embodiments, controller 72 determines the amount of movement toward the sides or ends of the patient support apparatus that trigger an exit alert by calculating relative changes in weight on force sensors 74. In such embodiments, force sensors 74 are positioned at different locations (such as adjacent four corners of the support apparatus) and exit detection system 52 triggers an exit alert when the ratio of forces detected on one end versus the other end, or the ratio of forces detected on one side versus the other side, change by more than a threshold. In these embodiments, the threshold changes based on the user-selected sensitivity. That is, if the user selects a mode of operation with the most sensitivity, then the smallest preprogrammed threshold is selected, and relatively small changes in the ratio of forces triggers an exit alert. If the user selects a mode of operation having a medium sensitivity level, a higher threshold than that used with the highest sensitivity level is used. And if the user selects a mode of operation having a high sensitivity level, the highest preprogrammed threshold level is used.

In other embodiments, such as will be described in more detail below, exit detection system 52 uses force sensors 74 to determine a center of gravity of the occupant in order to determine if the occupant is about to exit patient support apparatus 20, and thus issue an exit alert. In these center-of-gravity determining embodiments, exit detection system 52 may be configured to determine the center of gravity of the patient using the system and method disclosed in commonly assigned U.S. Pat. No. 5,276,432 issued to Travis and entitled PATIENT EXIT DETECTION MECHANISM FOR HOSPITAL BED, the complete disclosure of which is incorporated herein by reference. In other embodiments, other algorithms for determining the center of gravity may be used.

As will be discussed in greater detail below, exit detection system 52 is also configured to issue an exit alert based on drops in weight that exceed a threshold. That is, exit detection system 52 is configured to take a baseline weight reading of the patient, either when the patient initially enters the patient support apparatus 20 or when the exit detection system 52 is initially armed. Thereafter, exit detection system 52 repetitively monitors the weight detected by force sensors 74 and compares the monitored weight to the baseline weight reading. If controller 72 detects a drop in the current weight reading that is more than a threshold percentage of the baseline weight reading, controller 72 issues an exit alert. This issuance of the exit alert is independent of the center of gravity, or position, determination that controller 72 also performs. In other words, for example, in some embodiments, controller 72 is configured to issue an exit alert if the patient's center of gravity moves outside of the boundaries of an active zone or if the weight detected by the force sensors drops below a defined percentage of the baseline weight reading. These two conditions (1. center of gravity moving outside of an active zone, and 2. weight dropping by more than a threshold) are therefore independent of each other, and either one of them can result in controller 72 issuing the exit alert, as will be explained in greater detail below.

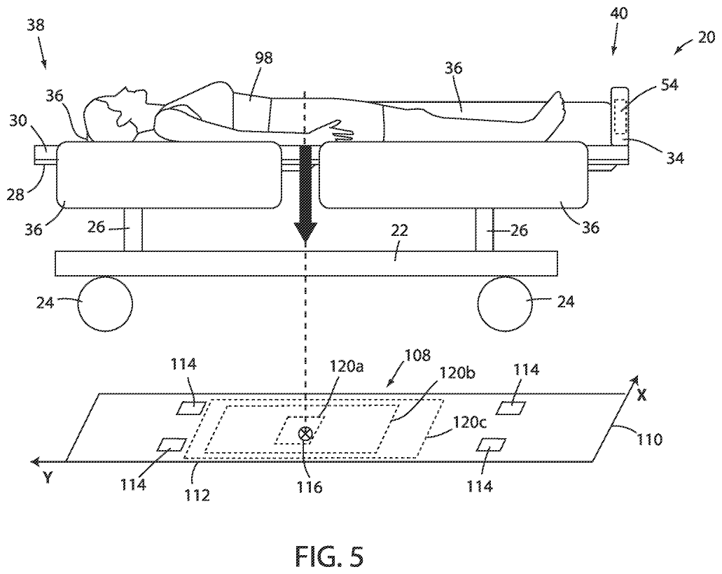

Turning first to the condition of monitoring the patient's center of gravity, FIG. 5 illustrates more clearly how controller 72 repetitively determines the center of gravity of the patient 98 in a planar coordinate frame of reference, such as reference frame 108. The plane of reference frame 108 is oriented generally parallel to the plane of litter frame 28 (e.g. it is horizontal when patient support apparatus 20 is supported on a horizontal surface and litter frame 28 is not tilted). Reference frame 108 includes an X-axis 110 and a Y-axis 112. X-axis 110 extends laterally in a direction generally parallel to a horizontal line extended from one side of the footboard 34 to the other side of the footboard 34 of patient support apparatus 20. Y-axis 112 extends longitudinally in a direction generally parallel to a line extending from head end 38 toward foot end 40 of patient support apparatus. Other frames of reference and/or coordinate systems can be used. Regardless of which frame of reference and/or coordinate system is used, exit detection controller 72 knows the location of force sensors 74 in the particular frame of reference and coordinate system used by the patient support apparatus 20. In the example shown in FIG. 5, force sensors 74 are shown in known locations 114.

In the illustrative example shown in FIG. 5, controller 72 has determined the occupant's center of gravity to be at a location 116. When exit detection system 52 is armed, controller 72 compares this center of gravity 116 to one of multiple alert zones 120a-c. The particular alert zone 120a-c to which the patient's center of gravity 116 is compared depends upon which sensitivity level the user has selected, as will be discussed in more detail below. Each zones 120a-c is defined in reference frame 108 and controller 72 determines whether the center of gravity 116 is inside or outside of whichever alert zone 120 is active (i.e. whichever one corresponds to the sensitivity level selected by the user). If center of gravity 116 moves outside of the active alert zone 120, controller 72 issues an alert indicating that the occupant is about to exit from patient support apparatus 20. The boundaries of alert zones 120a-c therefore define thresholds for the degree of movement of a patient that will trigger an exit alert by exit detection system 52.

When determining whether the center of gravity 116 is outside or inside of the active alert zone 120, controller 72 may first compute the center of gravity in a first one of the directions of coordinate frame of reference 108 (X direction or Y direction), compare that value to the corresponding boundaries of the zone in that particular direction and, if it is inside the boundaries, compute the center of gravity in the other direction of coordinate frame of reference 108 (X direction or Y direction). Alternative methods for determining whether the patient's current center of gravity 116 is currently within or outside of the active zone 120 may also be used.

As shown in FIG. 5, there are three different alert zones 120a, b, and c. Alert zones 120a, b, and c have different sizes, allowing the occupant to engage in different amounts of movement prior to triggering an exit alert. A user selects which one of the alert zones 120a-c will be the active alert zone using control panel 54. As discussed more below, this selection may be carried out by touching (or otherwise activating) one of the three sensitivity controls 122a-c shown on exit detection system arming screen 100 (see FIG. 4). After a user has armed exit detection system 52 and selected the desired sensitivity level, controller 72 repetitively recalculates the occupant's center of gravity 116 based upon the outputs from force sensors 74 and compares the calculated center of gravity 116 to the active zone. If the center of gravity 116 is within the active alert zone 120, no exit alert is issued. If the center of gravity 116 moves outside of the active zone 120, controller 72 issues an alert. In some embodiments, in order to avoid issuing an alert based upon transient weight signals shifting the center of gravity 116 outside of the active zone 120 for a fleeting moment, controller 72 only issues an alert if the center of gravity 116 moves outside of the active zone 120 for more than a threshold amount of time (which may be on the order of seconds or a fraction of a second).

In the example shown in FIG. 5, alert zone 120a has the smallest area and corresponds to the most sensitive level for exit detection system 52. Alert zone 120b has a larger area than zone 120a and corresponds to a medium sensitivity level for exit detection system 52. Alert zone 120c has the largest area of all of the zones 120 and corresponds to the least sensitive level. The size of the alert zones 120 in frame of reference 108 therefore corresponds to the amount of freedom that the patient has to move toward the sides and/or ends of patient support apparatus 20 before an exit alert is issued by exit detection system 52.

In at least one embodiment, a user is able to select which alert zone 120a-c exit detection system 52 uses for determining whether or not to issue an exit alert (i.e. the active zone) by pressing on one of the three sensitivity controls 122a-c shown on display 80 of control panel 54 (see FIG. 4). If the user selects the high sensitivity control 122a, exit detection system 52 uses zone 120a as the active zone. If the user selects medium sensitivity control 122b, exit detection system 52 uses zone 120b as the active zone. And if the user selects low sensitivity control 122c, exit detection system 52 uses zone 120c as the active zone. Control 122a includes a graphical representation of a person partially sitting up on patient support apparatus 20. Control 122b includes a graphical representation of a person sitting up more than control 122a and with one or his or her legs partially positioned over the side of patient support apparatus. Control 122c includes a graphical representation of a person facing the edge of the support surface (mattress) with their legs over the edge of the support surface and with their arms resting on their legs.

It will be understood by those skilled in the art that the particular size and shape of the alert zones 120a-c shown in FIG. 5 are merely illustrative examples, and that the size and shape of these alert zones may vary in different embodiments. Further, as will be discussed more below, in some embodiments, patient support apparatus 20 may be configured to dynamically adjust the size and/or shape of these alert zones 120 during operation of patient support apparatus 20, such as when one or more components on patient support apparatus 20 move to a different position and/or orientation that indicate a greater or lesser likelihood of a patient exiting (e.g. siderails 36 are raised or lowered); when one or more components on patient support apparatus 20 move in a manner that changes the center of gravity calculation (e.g. litter frame 28 changes its tilt); when an object is added or removed from litter frame 28; and/or when one or more other changes are made that desirably lead to different size and/or shaped zones 120 (e.g. when patient support apparatus 20 is configured with a variable width support deck 30 and the width of support deck 30 is changed). Still other factors may be used to dynamically adjust the boundary of one or more zones 120.

Depending upon which sensitivity level the user selects, control panel 54 is configured to display the selected zone control 122 in a different manner than the other zone controls 122 on the exit detection system arming screen 100 (FIG. 4). This different manner may involve displaying the selected (i.e. active) zone in a different color; highlighting the selected zone; changing a background of the selected zone; adding a check mark or some other graphic to indicate the selection; and/or some other changes or combination of changes that visually identify which zone is the currently selected zone 120. In the example shown in FIG. 4, the user has selected the medium sensitivity level and control panel 54 indicates this selection by displaying sensitivity control 122b with is different background color than it displays sensitivity controls 122a and 122c.

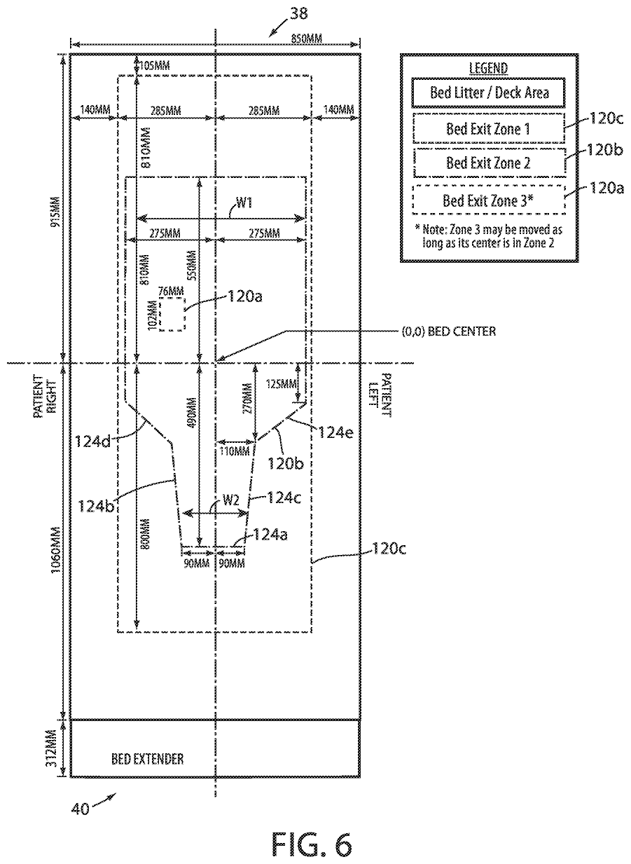

In at least one embodiment, controller 72 is configured to implement zones 120a-c in the manner illustrated in FIG. 6. As shown therein, the most sensitive zone 120a has a generally rectangular shape that has the smallest area of all of the zones 120a-c. In some embodiments, zone 120a is defined at a location that coincides with the patient's calculated center of gravity at the moment exit detection system 52 is initially armed. Controller 72 uses zone 120a for comparing the patient's center of gravity to when the user arms exit detection system 52 with its highest sensitivity level (via control 122a of FIG. 4). Zone 120b has a larger area than zone 120a and is used by controller 72 when the user selects the middle sensitivity level for arming exit detection system 52 (via control 122b of FIG. 4). Zone 120c has the largest area and is used by controller 72 when the user selects the least sensitive level for arming exit detection system 52 (i.e. via control 122c in FIG. 4).

As can be seen in FIG. 6, zone 120b is an eight sided rectilinear shape. Towards head end 38, zone 120b has a first width W1. Towards foot end 40, zone 120b has a second width W2 that is smaller than width W1. Therefore the patient's center of gravity is permitted less lateral leeway toward foot end 40 than it is toward head end 38 before it triggers an exit alert. This shape may be implemented because a patient typically does not exit patient support apparatus 20 from head section 44 (aka Fowler section 44), but instead more often tends to exit from seat section 46 and/or foot section 46. Thus, lateral movement of the patient's center of gravity in Fowler section 44 is less likely to indicate patient exit, and zone 120b therefore allows such greater lateral movement without triggering an exit alert.

It will be understood that, although FIG. 6 shows various dimensions for the segments of zones 120a-c, these dimensions may be changed in different embodiments. It will also be understood that the size and shape of zone 120b may be changed from what is shown in FIG. 6. For example, instead of having eight sides, zones 120b may have a boundary of only seven sides. In one such example, foot end segment 124a is eliminated, and the two lateral segments 124b and c merge together at a point. In another example, segments 124d and 124e merge together at a point and segments 12a-c are omitted. Still other variations are possible.

It will also be understood that various other modifications to zone 120b can be made. For example, instead of a rectilinear shape, zone 120b may be modified so that one or more its straight segments 124 are replaced with arced segments, or other types of curved segments. Still further, in some embodiments, controller 72 is configured to adjust the size, shape, and/or location of zone 120b (and/or zone 120c) in response to movement of one or more components of patient support apparatus 20. In some embodiments, exit detection system 52 may also implement any of the features disclosed in commonly assigned U.S. patent application Ser. No. 17/318,476 filed May 12, 2021, by inventors Sujay Sukumaran et al. and entitled PATIENT SUPPORT APPARATUS WITH AUTOMATIC EXIT DETECTION MODES OF OPERATION, the complete disclosure of which is incorporated herein by reference.

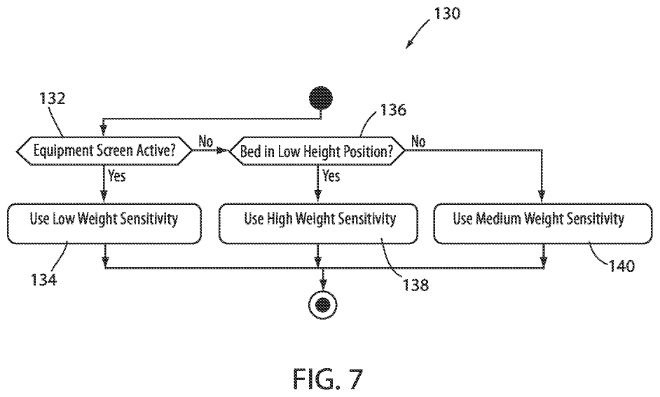

As was mentioned previously, controller 72 is also configured to issue an exit alert (when exit detection system 52 is armed) if controller 72 detects a drop in weight on patient support apparatus 20 that exceeds a threshold. As will now be discussed, in some embodiments, controller 72 is configured to vary the threshold based on the state of one or more components of patient support apparatus 20. In some embodiments, controller 72 varies the threshold for only one of the selected sensitivity levels and uses a fixed threshold for the other two sensitivity levels, while in other embodiments, controller 72 is configured to vary the sensitivity level for two or more of the sensitivity levels. One example of this is shown in more detail in FIG. 7.

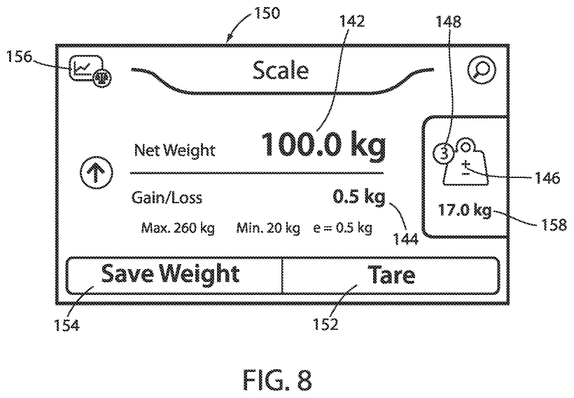

FIG. 7 illustrates a flow chart of a variable weight algorithm 130 that controller 72 is configured to implement in at least one embodiment of patient support apparatus 20. As shown therein, controller 72 is configured to use a low weight sensitivity when a user has activated an equipment log screen, to use a high weight sensitivity when the litter frame is in its lowest height, and to use a medium weight sensitivity in other situations. More specifically, at step 132, controller 72 determines if an equipment screen, also referred to as a weight log screen, or equipment log screen, is currently being displayed on display 80. In some embodiments of patient support apparatus 20, controller 72 is configured to display such an equipment weight log screen in response to a user pressing on weight log control 146 (see FIG. 8). Other manners may be implemented for allowing a user to navigate to the equipment weight log screen. Controller 72 is apprised of the state of the equipment screen via sensor 64 (FIG. 2), which, in some embodiments, may be a touch sensor incorporated into a conventional touchscreen that is adapted to detect when a user presses on a control for controlling the equipment log screen. Other types of sensors may also be used.

The equipment weight log screen is a screen that allows a user to add or remove weight from patient support apparatus 20 in a manner that keeps the weight gain or weight loss separate from the patient's weight. In other words, the equipment weight log screen allows controller 72 to differentiate weight that is being added or removed from patient support apparatus 20—due to objects or other equipment being added or removed-separate from the patient's weight. Thus, for example, if a user wishes to add a ten pound piece of equipment to patient support apparatus 20, but doesn't want patient support apparatus 20 to attribute that ten pound weight to the patient's weight, he or she can access the equipment weight log screen, add the ten pound weight, and controller 72 is configured to add the ten pound weight to an internal weight log that keeps track of the total weight of equipment onboard patient support apparatus 20. When it comes time to determine the patient's weight, controller 72 subtracts the value of the weight in this internal weight log from the total weight readings from force sensors 74 (as well as the tare weight) to arrive at the weight of the patient. Accordingly, the equipment weight log screen allows the user to make changes to the internally maintained equipment weight log, thereby updating the record (i.e. log) of what weight on patient support apparatus 20 is due to non-patient objects.

When controller 72 executes algorithm 130 (FIG. 7), it checks to see if the equipment weight log screen (or other control) is active at step 132. If it is, controller 72 proceeds to step 134 and uses a low weight sensitivity for exit detection system 52. In some embodiments, the low weight sensitivity may be on the order of 20% to 40% of the baseline weight, although other thresholds may be used. The baseline weight, as noted, is captured automatically by controller 72 when exit detection system 52 is armed, and/or after the patient enters patient support apparatus 20. When exit detection system 52 uses the low weight sensitivity, controller 72 issues an exit alert whenever it detects that the weight onboard patient support apparatus 20 has dropped to a level less than a low weight threshold. Thus, if the low sensitivity weight threshold is, say, 40%, and the patient weighs one hundred pounds, controller 72 will issue an exit alert whenever the patient weight detected by exit detection system 52 drops to forty pounds or less.

If controller 72 determines at step 132 that the equipment log screen is not active, it moves to step 136. At step 136, controller 72 determines whether the litter frame has been moved to a low height position. The low height position may refer to the single lowest height that litter frame 28 can be moved to, or it may refer to a range of low heights that litter frame 28 can be moved to. The height of litter frame 28 is reported to controller 72 from height sensor 62. Regardless of whether algorithm 130 looks at the single lowest height, or a range of low height values, if controller 72 determines that the current height of litter frame 28 is at a position considered low height, it moves to step 138 of algorithm 130 (FIG. 7). At step 138, controller 72 implements a high weight sensitivity for exit detection system 52.

When implementing a high weight sensitivity for exit detection system 52 at step 138, controller selects a higher percentage of the baseline weight and looks to see if the currently measured (and repetitively measured) weight has dropped below that higher percentage of the baseline weight reading. In some embodiments, the high weight sensitivity may be on the order of 60% to 80% of the baseline weight, although other thresholds may be used. The baseline weight, as noted, is captured automatically by controller 72 when exit detection system 52 is armed, and/or after the patient enters patient support apparatus 20. When exit detection system 52 uses the high weight sensitivity, controller 72 issues an exit alert whenever it detects that the weight onboard patient support apparatus 20 has dropped to a level less than the high weight threshold. Thus, if the high sensitivity weight threshold is, say, 70%, and the patient weighs one hundred pounds, controller 72 will issue an exit alert whenever the patient weight detected by exit detection system 52 drops to seventy pounds or less.

If controller 72 determines at step 136 that litter frame 28 is not currently in a low height position, it moves to step 140 of algorithm 130 (FIG. 7). At step 140, controller 72 uses a medium weight sensitivity for exit detection system. In some embodiments, the medium weight sensitivity refers to a percentage of the baseline weight reading of around 50%. Thus, when exit detection system 52 uses a medium weight sensitivity, controller 72 issues an exit alert whenever it detects that the weight onboard the patient support apparatus 20 has dropped to a level less than the medium weight threshold. If the medium sensitivity weight threshold is, say, 50%, and the patient weighs one hundred pounds, controller 72 will issue an exit alert whenever the patient weight detected by exit detection system 52 drops to fifty pounds or less.

It will be understood that a number of modifications can be made to the form of algorithm 130 shown in FIG. 7. For example, in some embodiments of patient support apparatus 20, controller 72 is configured to implement algorithm 130 only for a single sensitivity level of exit detection system 52. For example, in such embodiments, controller 72 may be configured to implement algorithm 130 only when the user selects the medium sensitivity level control 122b (FIG. 4). In such embodiments, controller 72 may be configured to use a fixed weight threshold if the user selects the high sensitivity level (via control 122a) or the low sensitivity level (via control 122c). For the high and low sensitivity levels selected via controls 122a and 122c, controller 72 may issue the exit alert if the patient's weight drops below a threshold that remains that same, regardless of the state of the equipment log screen and/or the height of litter frame 28. In some such embodiments, this fixed weight threshold may be on the order of 50% of the baseline reading, although other fixed values may be used. In other embodiments, controller 72 may be configured to use algorithm 130 for two or more of the sensitivity levels that the user selects via screen 100 (FIG. 4).

It will also be understood that, although algorithm 130 of FIG. 7 only discloses changing the weight sensitivity of exit detection system 52 based on the state of the equipment log screen and the height of litter frame 28, algorithm 130 may be modified to vary the weight sensitivity based on other, and/or additional factors. For example, in some embodiments, controller 72 may vary the weight sensitivity used by exit detection system 52 based on the state of one or more siderails 36 (raised, lowered, or intermediate positions), the state of the brake, the state of the Fowler angle, a time of day, a location of the patient support apparatus 20 within a healthcare facility, a fall risk rating or other characteristics of a patient, etc.

As was noted previously, in some embodiments, controller 72 is configured to issue an exit alert when exit detection system 52 is armed if either of two conditions are met: (1) if the patient's center of gravity moves outside of the active zone, or (2) if the patient's weight drops by more than a threshold, which, as noted above in the discussion of FIG. 7, may vary depending upon the particular state of one or more of the components of patient support apparatus 20. In other embodiments, exit detection system 52 may be configured to issue an exit alert based only on a weight drop that exceeds a variable threshold, and to not issue exit alerts based on the patient's center of gravity. Still further, in some embodiments, exit detection system 52 may be configured to monitor both the patient's center of gravity and a weight drop when the user selects a particular one (or ones) of the sensitivity levels via 122a-c, and to monitor only one or these conditions (center of gravity or weight drop) when the user selects a different one (or ones) of the sensitivity levels via controls 122a-c.

It will be understood that the threshold for issuing the exit alert based on a reduction in the detected patient weight is based on a reduction in weight that occurs over a relatively short time (e.g. within seconds), not a reduction in weight that may occur over the course of days or weeks. In other words, the threshold is based on a reduction in the patient weight that would be expected to occur in the time span it might take a patient to climb out of patient support apparatus 20.

As was noted above, exit detection system 52 may be configured to change the size, shape, and/or position of one or more of alert zones 120a-c in any of the manners that are disclosed in commonly assigned U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES, and Ser. No. 15/266,575 filed Sep. 15, 2016, by inventors Anuj K. Sidhu et al. and entitled PERSON SUPPORT APPARATUSES WITH EXIT DETECTION SYSTEMS, the complete disclosures of both of which are incorporated herein by reference. Any of the adjustments disclosed in these patent applications may be made by exit detection system 52.

As an alternative to making adjustments to the size, shape, and/or position of one or more alert zones 120, exit detection system 52 may be configured to adjust the calculated center of gravity 116 of the patient to take into account that portion of the change in the center of gravity 116 that is due to the movement of the component(s) of the patient support apparatus 20. In these embodiments, exit detection system 52 may adjust the center of gravity 116 so that the effects of the component(s) movement on the center of gravity calculation 116 are stripped out of the patient's center of gravity calculation. Several manners in which these adjustments to the center of gravity can be made are disclosed in commonly assigned U.S. Pat. No. 10,617,327 issued Apr. 14, 2020, to inventors Marko Kostic et al. and entitled EXIT DETECTION SYSTEM WITH COMPENSATION, the complete disclosure of which is incorporated herein by reference. The adjustments to the patient's calculated center of gravity may be made for movement of any of the actuators 26a-b, 58a-b, and/or for movement of other components of patient support apparatus 20.

In still other embodiments, exit detection system 52 is configured to not only adjust the size, shape, and/or position of one or more alert zones, it is also configured to adjust the patient's calculated center of gravity. Thus, for example, if the Fowler tilt angle increases during movement, the readings from each of the force sensors 74 are adjusted based on the known weight of the patient, and the known distribution of that weight on the force sensors 74 prior to the movement of the Fowler section (it is assumed that the patient does not move on the support deck during this movement). This adjustment changes the calculated center of gravity by effectively removing the shift in the patient's weight that is due to the raising of the Fowler (but does not remove shifts in the patient's weight due to his or her movement relative to the support deck—as discussed more in the aforementioned U.S. Pat. No. 10,617,327 patent). The adjustment to the side, shape, and/or alert zone is also implemented in order to allow the patient an adjusted area of alert-free movement that betters matches his or her new orientation after the Fowler has been raised).

FIG. 8 illustrates an example of a scale control screen 150 that may be displayed by controller 72 on display 80 in response to a user pressing on scale control 78b (FIG. 3). Controller 72 may also, or additionally, be configured to allow a user to navigate to scale control screen 150 in other manners. Scale control screen 150 includes a patient weight indicator 142, a gain/loss indicator 144, a weight log control 146, an object number 148, an object total weight indicator 158, a zero control 152, a save weight control 154, and a scale history control 156. Patient weight indicator 142 indicates the currently measured patient weight, which is determined by subtracting the weight of any objects in the weight log from the current total weight on force sensors 74 (after the force sensors have been zeroed). Gain/loss indicator 144 indicates the total patient weight loss or gain since the patient's weight was last saved by the user. Controller 72 is configured to store in memory 76 each of the patient weight readings that a caregiver saves for a particular patient. The gain/loss indicator displays the difference between the current patient weight and the last saved patient weight reading in memory 76.

Log control 146 is an icon that corresponds to the weight log discussed above with respect to algorithm 130. That is, log control 146 corresponds to the weight log that controller 72 maintains of all of the objects or equipment that may be added to the litter frame 28 of patient support apparatus 20. If the user presses on weight log control 146, controller 72 is configured to display a screen that provides more information about the weight log, such as the screens shown in FIGS. 8-11, 13, 15, and/or 16-18 of commonly assigned U.S. patent application Ser. No. 63/255,211 filed Oct. 13, 2021, by inventors Sujay Sukumaran et al. and entitled PATIENT SUPPORT APPARATUS WITH AUTOMATIC SCALE FUNCTIONALITY, the complete disclosure of which is incorporated herein by reference.

Object number 148 indicates the number of objects that are currently in the weight log. Thus, in the example shown in FIG. 8, the weight log currently has three objects recorded therein that are positioned on litter frame 28. Weight total indicator 158 indicates the total weight of the objects currently stored in the weight log. Thus, in the example of FIG. 8, the three objects stored in the weight log have a total cumulative weight of 17.0 kg, as indicated by indicator 158.

Screen 150 (FIG. 8) also includes a save weight control 154 and a zero control 152. If the user presses on the save weight control 154, controller 72 is configured to store the currently measured patient weight (as indicated by indicator 142) within memory 76. That stored patient weight is then used as a baseline for future patient weight readings when controller 72 computes the value to be shown by the gain/loss indicator 144. In other words, when the user presses the save weight control 154, controller 72 zeroes the gain/loss indicator 144. In the example of FIG. 8, after the user presses the save weight control 154, controller 72 would change indicator 144 to zero and thereafter only show non-zero values if the patient's weight deviated from 100.0 kg.

If the user presses on zero control 152 (FIG. 8), controller 72 is configured to zero (i.e. tare) the scale system. That is, in response to control 152 being activated by a user, controller 72 takes a snapshot of the current total weight readings recorded by force sensors 74 and considers that weight value to be equal to the empty weight of patient support apparatus 20 (i.e. the weight on the scale system when no patient is present and no objects of interest are present). Typically, a caregiver uses control 152 to zero the scale system when the patient is absent, but a mattress, a pillow, bedding, and other standard items (if any) are present on the patient support apparatus 20. In this manner, the weight of the mattress, pillow, bedding, etc., as well as the structural weight of the support deck 30 and litter frame 28, are zeroed out. In some embodiments, after the user has zeroed the scale system using control 152, controller 72 is configured to erase any items that were stored in the weight log.

Scale screen 150 also includes a scale history control 156. When a user presses on control 156, controller 72 is configured to display a different screen that graphically shows a history of the patient's weight readings. The graph may have time on the X-axis and the patient's weight on the Y-axis. The patient weight history screen gives the caregiver a visual overview of the fluctuations in the patient's weight while they were assigned to that particular patient support apparatus 20. In some embodiments, scale history control 156 is displayed in a first color (and/or with a first configuration) when there is data contained within the scale history, and in a second color (and/or with a second configuration) when there is no data contained within the scale history. Thus, for example, if the caregiver has never taken a weight reading of the patient, controller 72 might display control 156 with a first color until the caregiver takes a first patient weight reading, at which point controller 72 will switch to displaying control 156 in a second color so that the user knows that previous patient weight readings have been taken.

In some embodiments, equipment log sensor 64 (FIG. 2) is configured to detect when a user presses on weight log control 146 (FIG. 8). Equipment log sensor 64 forwards this detection to controller 72 and controller 72, as discussed, uses this information in step 132 of algorithm 130. In other words, at step 132, controller 72 checks to see if the user has activated the weight log control 146 on FIG. 8 or not and, if so, proceeds to step 134, and if not, proceeds to step 136.

It will also be understood that, although exit detection system 52 has been described herein as primarily being controlled via control panel 54, patient support apparatus 20 may be configured to allow exit detection system 52 to be controlled via one or more portable electronic devices 92 (FIG. 2). Such devices may be configured to display the same, or similar, screens on their displays 94 as those shown and described herein as being displayed on display 80, thereby presenting the user of the portable computing device 92 with the same options and screens that he or she generally sees on display 80 of control panel 54. In this manner, there is little visual difference to the user between controlling exit detection system 52 via control panel 54 and via a mobile electronic device 92. As was noted previously, such mobile electronic devices 92 may be further configured to control other aspects of patient support apparatus 20, and/or to carry out other functions for assisting the caregiver.

Although exit detection system 52 has been primarily described herein as computing a center of gravity 116 of the occupant and comparing the position of the computed center of gravity to an active zone 120, it will be understood by those skilled in the art that exit detection system 52 can be modified to process the outputs of force sensors 74 in other manners besides computing a center of gravity 116. For example, controller 72 may be configured to sum the total amount of force on force sensors 74 when patient support apparatus 20 is occupied and then looks for shifts of more than a threshold amount of that weight to a side, head end, or foot end of patient support apparatus. For example, if a 100 kilogram person is occupying patient support apparatus 20, exit detection system 52 may be modified to trigger an exit alert if more than X percent, say, 70% (0.70×100=70 kilograms) of the total forces are detected by the two force sensors 74 positioned along the right side of patient support apparatus 20, or by the two force sensors 74 positioned along the left side of patient support apparatus 20. In some embodiments, a different ratio of the forces detected by the two force sensors 74 positioned along the foot end 40 of patient support apparatus 20 may trigger an exit alert if the ratio exceeds a different threshold, while still another ratio of the forces detected by the two force sensors 74 positioned along the head end 38 of patient support apparatus 20 may trigger an exit alert if that ratio exceeds yet a different threshold. In sum, exit detection system 52 can be modified to compute one or more ratios of the force detected by a first force sensor 74 (or the sum of forces detected by a combination of first force sensors 74) to the force, or sum of forces, detected by at least one other force sensor 74. The one or more ratios may then be compared to one or more thresholds for determining whether to issue an exit alert or not. Other types of weight distribution changes may also be used to trigger an exit alert.

As was previously noted, controller 72 of exit detection system 52 may be further configured to change the boundary of the zone(s) 120 in response to a variety of different triggers. These triggers includes, but are not limited to, movement of components for which real time position feedback is not available (e.g. the raised/lowered position of siderail 36, a width of deck 30, a length of deck 30, etc.), a patient characteristic, the addition or removal of an object from litter frame 28, the implementation of a mattress therapy (e.g. lateral rotation), or still other factors. Several manners in which the zones 120a-c may be adjusted in response to a patient characteristic are disclosed in the commonly assigned U.S. patent application Ser. No. 15/266,575, which has already been incorporated herein by reference. Further, methods for automatically identifying the addition or removal of a non-patient object on a patient support apparatus using force sensors 74 are disclosed in commonly assigned U.S. Patent Application Publication No. 2016/0022218 to Hayes et al., entitled PATIENT SUPPORT APPARATUS WITH PATIENT INFORMATION SENSORS, the complete disclosure of which is hereby incorporated herein by reference. In general, any one of the triggers for adjusting a boundary of an alert zone disclosed in commonly assigned 62/889,254 application (incorporated by reference previously herein) may be used by exit detection system 52 to adjust one or more of the boundaries of zones 120a-c.

It will also be understood that all of the aforementioned adjustments to alert zones 120a-c and/or to the patient's calculated center of gravity are accomplished based on readings taken from force sensors 74 that are, in at least one embodiment, automatically adjusted in order to compensate for errors introduced into these sensor readings from litter frame 28 being non-level. That is, in some embodiments, force sensors 74 are load sensors whose outputs do not reflect the true load placed thereon when the load applied to the load cell is tilted, such as may happen when litter frame 28 is tilted out of a horizontal orientation. In such cases, the level of tilt is detected by one or more sensors onboard patient support apparatus 20 and a simple trigonometric calculation (based on the detected tilt angle) is applied to the outputs of the load cells 74 to remove this error in the load measurement. These tilt-adjusted load cell readings are then processed and used to compute the center of the gravity of the patient and/or the distribution of the patient's weight, along with the concomitant changes, as appropriate, to the alert zones 120a-c discussed above.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a litter frame;
a support surface supported by the litter frame and adapted to support a patient thereon;
a plurality of force sensors adapted to detect downward forces exerted on the support surface;
a control panel adapted to allow a user to arm an exit detection function;
a sensor adapted to detect a state of component of the patient support apparatus; and
a controller adapted, when the exit detection function is armed, to repetitively determine a current weight supported on the support surface using outputs from the plurality of force sensors, to select a variable threshold based on an output from the sensor, to compare the current weight to the variable threshold, and to issue an exit alert if the current weight is less than the variable threshold.

2. The patient support apparatus of claim 1 wherein the controller is further adapted to determine a baseline weight reading when the patient is supported on the patient support apparatus using outputs from the plurality of force sensors, and to select the variable threshold by selecting a percentage of the baseline weight reading.

3. The patient support apparatus of claim 1 further comprising a lift subsystem adapted to raise and lower the litter frame, and wherein the output of the sensor is indicative of a height of the litter frame, the controller is adapted to select a first value for the variable threshold when the height of the litter frame is at a low height and to select a second value for the variable threshold when the height of the litter frame is at a high height, wherein the first value is higher than the second value.

4. (canceled)

5. The patient support apparatus of claim 1 further comprising an equipment change control adapted to be activated by a user in order to update an onboard log of equipment weight, wherein the controller is adapted to use the onboard log of equipment weight to keep track of which component of the current weight belongs to the patient and which component belongs to equipment.

6. The patient support apparatus of claim 5 wherein the output of the sensor is indicative of the user activating the equipment change control.

7. The patient support apparatus of claim 6 wherein the controller is adapted to select a first value for the variable threshold when the equipment change control have been activated and to select a second value for the variable threshold when the equipment change control has not been activated, wherein the first value is less than the second value and wherein the equipment change control includes an icon on a touchscreen display and the sensor is a touch sensor.

8. (canceled)

9. The patient support apparatus of claim 1 further comprising a sensitivity control adapted to allow the user to select a first sensitivity level or a second sensitivity level for issuing the exit alert, wherein the controller is adapted to compare the current weight to the variable threshold when the user has selected the first sensitivity level, but not when the user has selected the second sensitivity level.

10. The patient support apparatus of claim 9 wherein the controller is adapted to compare the current weight to a fixed threshold when the user has selected the second sensitivity level, and the controller is adapted to issue the exit alert if the current weight is less than the fixed threshold and the user has selected the second sensitivity level.

11. The patient support apparatus of claim 1 wherein the controller is further adapted to repetitively calculate a center of gravity of the patient using outputs from the plurality of force sensors, to repetitively compare the calculated center of gravity to a zone, and to issue the exit alert if the calculated center of gravity moves outsides of a boundary of the zone, and wherein the boundary of the zone is a rectilinear boundary having at least seven sides.

12. (canceled)

13. The patient support apparatus of claim 11 wherein the controller is further adapted to repetitively calculate a center of gravity of the patient using outputs from the plurality of force sensors, to repetitively compare the calculated center of gravity to a zone, and to issue the exit alert if the calculated center of gravity moves outsides of a boundary of the zone, wherein the boundary of the zone defines a shape having a first width defined toward a head end of the patient support apparatus and a second width defined toward a foot end of the patient support apparatus, and the first width is greater than the second width.

14. A patient support apparatus comprising:
a litter frame;
a support surface supported by the litter frame and adapted to support a patient thereon;
a plurality of force sensors adapted to detect downward forces exerted on the support surface;
a control panel adapted to allow a user to arm an exit detection function;
a sensitivity control adapted to allow the user to select a first sensitivity level or a second sensitivity level for issuing an exit alert when the exit detection function is armed; and
a controller adapted to, when the exit detection function is armed and the first sensitivity level is selected, perform the following:
(a) repetitively determine a current weight supported on the support surface using outputs from the plurality of force sensors;
(b) compare the current weight to a first threshold; and
(c) issue the exit alert if the current weight is less than the first threshold;
wherein the controller is further adapted to, when the exit detection function is armed and the second sensitivity level is selected, perform the following:
(i) repetitively determine the current weight supported on the support surface using outputs from the plurality of force sensors;
(ii) compare the current weight to a second threshold; and
(iii) issue the exit alert if the current weight is less than the second threshold, wherein the first threshold is different from the second threshold.

15. The patient support apparatus of claim 14 further comprising a sensor adapted to detect a state of a component of the patient support apparatus, and wherein the controller is further adapted to vary the first threshold based on an output of from the sensor.

16. The patient support apparatus of claim 15 further comprising a lift subsystem adapted to raise and lower the litter frame, and wherein the output of the sensor is indicative of a height of the litter frame, the controller is adapted to select a first value for the first threshold when the height of the litter frame is at a low height and to select a second value for the first threshold when the height of the litter frame is at a high height, wherein the first value is higher than the second value.

17. (canceled)

18. The patient support apparatus of claim 15 further comprising an equipment change control adapted to be activated by the user in order to update an onboard log of equipment weight, wherein the controller is adapted to use the onboard log of equipment weight to keep track of which component of the current weight belongs to the patient and which component of the current weight belongs to equipment.

19. The patient support apparatus of claim 18 wherein the output of the sensor is indicative of the user activating the equipment change control.

20. The patient support apparatus of claim 19 wherein the controller is adapted to select a first value for the first threshold when the equipment change control have been activated and to select a second value for the first threshold when the equipment change control has not been activated, wherein the first value is less than the second value.

21. The patient support apparatus of claim 20 wherein the equipment change control includes an icon on a touchscreen display and the sensor is a touch sensor.

22. The patient support apparatus of claim 14 wherein the controller is further adapted to determine a baseline weight reading when the patient is supported on the patient support apparatus using outputs from the plurality of force sensors, and to select the first threshold by selecting a percentage of the baseline weight reading.

23. The patient support apparatus of claim 14 wherein the controller is further adapted to repetitively calculate a center of gravity of the patient using outputs from the plurality of force sensors and, when the exit detection function is armed and the first sensitivity level is selected, perform the following:
(d) repetitively compare the calculated center of gravity to a first zone, and
(e) issue the exit alert if the calculated center of gravity moves outsides of a boundary of the first zone; and
wherein the controller is further adapted to, when the exit detection function is armed and the second sensitivity level is selected, perform the following:
(iv) repetitively compare the calculated center of gravity to a second zone, and
(v) issue the exit alert if the calculated center of gravity moves outside of a boundary of the second zone, and
wherein the boundary of the first zone is a rectilinear boundary having at least seven sides, and the boundary of the second zone is a rectangle.

24. (canceled)

25. The patient support apparatus of claim 23 wherein the boundary of the first zone defines a shape having a first width defined toward a head end of the patient support apparatus and a second width defined toward a foot end of the patient support apparatus, and the first width is greater than the second width.
